SEQUENCE LISTING (4 sequences)

SEQ ID NO: 1 - protein, 551 aa, Homo sapiens
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 2 - protein, 548 aa, Homo sapiens
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 3 - protein, 482 aa, Homo sapiens
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 4 - protein, 448 aa, Homo sapiens
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQRPPDPAPAPLGAPGLPNGLLSGDEDFSSIADMDFSALLSQISS

(19) United States
(12) Patent Application Publication
(10) Pub. No.: US 2017/0157155 A1
(43) Pub. Date: Jun. 8, 2017

(54) METHOD OF TREATMENT OF NASOPHARYNGEAL CANCER

(71) Applicant: Millennium Pharmaceuticals, Inc., Cambridge, MA (US)

(72) Inventors: Stephen J. Blakemore, Littleton, MA (US); Alessandra M. Di Bacco, Newton, MA (US); George J. Mulligan, Lexington, MA (US)

(21) Appl. No.: 15/358,400

(22) Filed: Nov. 22, 2016

Related U.S. Application Data
(63) Continuation of application No. 14/373,985, filed on Jul. 23, 2014, now abandoned.
(60) Provisional application No. 61/590,115, filed on Jan. 24, 2012.

(51) Int. Cl. A61K 31/69; G01N 33/574; A61K 9/00

(52) U.S. Cl. CPC A61K 31/69; A61K 9/0019; A61K 9/0053; G01N 33/57407; G01N 2333/47

(57) ABSTRACT

The present invention provides a method of treating nasopharyngeal cancer with a proteasome inhibitor of formula (7). The invention also provides a method of treating a patient with nasopharyngeal cancer based on elevated expression levels of NFkB, as measured by a H-score of the patient's nasopharyngeal cancer tumor sample using a “NFkB p65 IHC assay. The invention also provides a method of determining whether to treat a patient with the compound of formula (/) based on the level of NFkB p65 in the patient's nasopharyngeal cancer tumor sample.

### PRIORITY APPLICATION

This application is a continuation of U.S. application Ser. No. 14/373,985, filed Jul. 23, 2014, which is a 35 U.S.C. §371(c) U.S. National Phase of PCT/US2013/022762, filed Jan. 23, 2013, which claims priority to U.S. Provisional Application No. 61/590,115 filed on Jan. 24, 2012. The entire contents of the foregoing applications are incorporated herein by reference.

### SEQUENCE LISTING

This application contains a Sequence Listing which is submitted herewith in electronically readable format. The electronic Sequence Listing file was created on Jan. 22, 2013, is named “sequencelisting.txt” and has a size of 16.8 kb (17,249 bytes). The entire contents of the Sequence Listing in the electronic sequencelisting.txt file are incorporated herein by this reference.

### FIELD OF THE INVENTION

The present invention provides a method of treating nasopharyngeal cancer with a proteasome inhibitor.

### BACKGROUND OF THE INVENTION

Nasopharyngeal cancer is a sub-type of head and neck cancer that arises from the epithelial cells that cover the surface and line the nasopharynx. The incidence of nasopharyngeal cancer has been reported at approximately 0.5 to 2 new cases per year per 100,000 in Europe and the USA. Rottey et al., Curr. Opin. Oncol., 23(3): 254-258 (2011). There are three subtypes of nasopharyngeal cancer recognized in the World Health Organization (WHO) classification: (i) Type 1—squamous cell carcinoma, typically found in the older adult population; (ii) Type 2 non-keratinizing carcinoma; and (iii) Type 3—undifferentiated carcinoma. Treatment for nasopharyngeal cancer often involves radiotherapy and/or chemotherapy. There remains a continuing need for new and improved treatments for patients with nasopharyngeal cancer. There remains a further need to identify nasopharyngeal patients most likely to benefit from treatment with a proteasome inhibitor.

Proteasome inhibition represents an important new strategy in cancer treatment. King et al., Science 274:1652-1659 (1996), describes an essential role for the ubiquitin-proteasome pathway in regulating cell cycle, neoplastic growth and metastasis. The authors teach that a number of key regulatory proteins, including cyclins, and the cyclin-dependent kinases p21 and p27^{KIP1}, are temporally degraded during the cell cycle by the ubiquitin-proteasome pathway. The ordered degradation of these proteins is required for the cell to progress through the cell cycle and to undergo mitosis.

The proteasome inhibitor VELCADE® (bortezomib; N-2-pyrazinecarbonyl-L-phenylalanine-L-leucineboronic acid) is the first proteasome inhibitor to achieve regulatory approval. Mitsiades et al., Current Drug Targets, 7:1341 (2006), reviews the clinical studies leading to the approval of bortezomib for the treatment of multiple myeloma patients who have received at least one prior therapy. Fisher et al., J. Clin. Oncol., 30:4867, describes an international multi-center Phase II study confirming the activity of bortezomib in patients with relapsed or refractory mantle cell lymphoma. Ishii et al., Anti-Cancer Agents in Medicinal Chemistry, 7:359 (2007), and Roccaro et al., Curr. Pharm. Biotech., 7:1341 (2006), discuss a number of molecular mechanisms that may contribute to the antitumor activities of bortezomib. The proteasome inhibitor MLN9708 [2,2′-{2-[(1R)-1-({[(2,5-dichlorobenzoyl)amino]acetyl}amino)-3-methylbutyl]-5-oxo-1,3,2-dioxaborolane-4,4-diyl}diacetic acid] is currently undergoing clinical evaluation for hematological and solid cancers. MLN9708 is a citrate ester which rapidly hydrolyzes to the active form [(1R)-1-({[(2,5-dichlorobenzoyl)amino]acetyl}amino)-3-methylbutyl]boronic acid (MLN2238) on exposure to aqueous solution or plasma. MLN9708 has demonstrated anti-tumor activity in a range of hematological and solid tumor xenograft models (Kupperman et al. (2010) Cancer Res. 70:1970-1980).

### SUMMARY

The invention relates to the discovery that patients with nasopharyngeal cancer respond to treatment with MLN9708. In one aspect, the invention relates to the discovery of the increased expression of Nuclear Factor Kappa-B RelA 65,000 dalton subunit (NFκB p65) in biological samples comprising cells obtained from patients with nasopharyngeal cancer and responsive to MLN9708.

Accordingly, the invention features treating nasopharyngeal cancer patients with MLN9708 if a sample from the patient demonstrates an elevated expression of NFκB p65.

### DESCRIPTION OF FIGURES

FIG. 1 shows H-scores as measured by an NFκB p65 IHC assay in head and neck cancer tumor samples as described in Example 2 below.

FIG. 2. FIG. 2 shows a multiple sequence alignment (Clustal W method) comparing the sequences of the isoforms of NFκB p65. NFκB p65 isoform 1 is SEQ ID NO: 1, NFκB p65 isoform 2 is SEQ ID NO:2, NFκB p65 isoform 3 is SEQ ID NO:3 and NFκB p65 isoform 4 is SEQ ID NO:4. An asterisk (*) beneath a residue in the alignment indicates that the residue is the same in all four isoforms. A dash (-) at a position in a sequence in the alignment indicates that the alignment does not place a residue from that sequence in that position occupied by a residue shown for another isoform in the alignment.

### DESCRIPTION OF THE INVENTION

The present invention provides a method for treating nasopharyngeal cancer, comprising administering a therapeutically effective amount of the compound of formula (I):

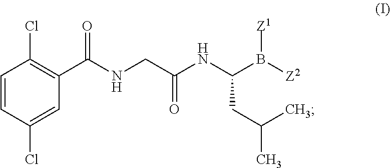

or a pharmaceutically acceptable salt or a pharmaceutical composition or a boronic acid anhydride thereof, wherein:
Z¹ and Z² are each independently hydroxy, alkoxy, aryloxy, or aralkoxy; or Z¹ and Z² together form a moiety derived from a boronic acid complexing agent.

The present invention provides a method for treating nasopharyngeal cancer, comprising administering a therapeutically effective amount of the compound of formula (I):

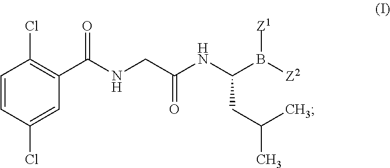

or a pharmaceutically acceptable salt or a pharmaceutical composition or a boronic acid anhydride thereof, wherein:
Z¹ and Z² are each independently hydroxy, alkoxy, aryloxy, or aralkoxy; or Z¹ and Z² together form a moiety derived from a boronic acid complexing agent;
to a patient whose nasopharyngeal cancer tumor sample is characterized by having an elevated level of NFκB p65.

In some embodiments, the method of treating nasopharyngeal cancer, comprises administering a therapeutically effective amount of the compound of formula (I):

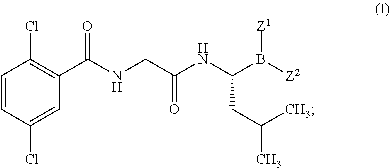

or a pharmaceutically acceptable salt or a pharmaceutical composition or a boronic acid anhydride thereof, wherein:
Z¹ and Z² are each independently hydroxy, alkoxy, aryloxy, or aralkoxy; or Z¹ and Z² together form a moiety derived from a boronic acid complexing agent;
to a patient whose nasopharyngeal cancer tumor sample is characterized by having a H-score of between 201 and 300, as measured by a NFκB p65 IHC assay.

In some embodiments, the method of treating nasopharyngeal cancer, comprises administering a therapeutically effective amount of the compound of formula (III-A):

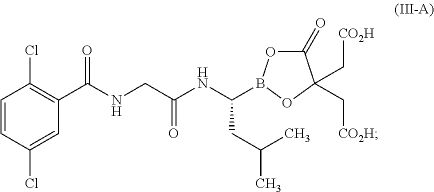 [SMILES: CC(C)C[C@H](NC(=O)CNC(=O)c1cc(Cl)ccc1Cl)B1OC(=O)C(CC(=O)O)(CC(=O)O)O1]

or a pharmaceutical composition thereof;
to a patient whose nasopharyngeal cancer tumor sample is characterized by having a H-score of between 201 and 300, as measured by a NFκB p65 IHC assay.

In another aspect, the present invention provides a method for determining whether to treat a patient with nasopharyngeal cancer with a compound of formula (I):

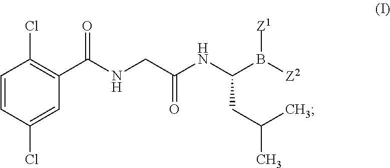

or a pharmaceutically acceptable salt or a pharmaceutical composition or a boronic acid anhydride thereof, wherein:
Z¹ and Z² are each independently hydroxy, alkoxy, aryloxy, or aralkoxy; or Z¹ and Z² together form a moiety derived from a boronic acid complexing agent; comprising:
a) measuring the level of NFκB p65 in a nasopharyngeal cancer tumor sample from the patient as an H-score, wherein the H-score is determined by a NFκB p65 IHC assay; and
b) determining to treat the patient with a therapeutically effective amount of the compound of formula (I) or a pharmaceutically acceptable salt or a pharmaceutical composition or a boronic acid anhydride thereof, if the nasopharyngeal cancer tumor sample is characterized by having a H-score of between 201 and 300, as measured by a NFκB p65 IHC assay.

The term “alkyl”, used alone or as part of a larger moiety, refers to a straight or branched chain or cyclic aliphatic group having from 1 to 12 carbon atoms. The term “alkoxy” refers to an —O-alkyl radical.

The terms “aryl” and “ar-”, used alone or as part of a larger moiety, e.g., “aralkyl”, “aralkoxy”, or “aryloxyalkyl”, refer to a C₆ to C₁₄ aromatic hydrocarbon, comprising one to three rings, each of which is optionally substituted. Preferably, the aryl group is a C₆₋₁₀ aryl group. Aryl groups include, without limitation, phenyl, naphthyl, and anthracenyl. An “aralkyl” or “arylalkyl” group comprises an aryl group covalently attached to an alkyl group, either of which independently is optionally substituted. Preferably, the aralkyl group is C₆₋₁₀ aryl(C₁₋₆)alkyl, C₆₋₁₀ aryl(C₁₋₄)alkyl, or C₆₋₁₀ aryl(C₁₋₃)alkyl, including, without limitation, benzyl, phenethyl, and naphthylmethyl.

The term “substituted”, as used herein, means that a hydrogen radical of the designated moiety is replaced with the radical of a specified substituent, provided that the substitution results in a stable or chemically feasible compound. Nonlimiting examples of suitable substituents include C₁₋₆ alkyl, C₃-8 cycloalkyl, C₁₋₆alkyl(C₃-s)cycloalkyl, C₂-8 alkenyl, C₂-8 alkynyl, cyano, amino, C₁₋₆ alkylamino, di(C₁₋₆)alkylamino, benzylamino, dibenzylamino, nitro, carboxy, carbo(C₁₋₆)alkoxy, trifluoromethyl, halogen, C₁₋₆ alkoxy, C₆₋₁₀ aryl, C₆₋₁₀ aryl(C₁₋₆)alkyl, C₆₋₁₀ aryl(C₁₋₆)alkoxy, hydroxy, C₁₋₆ alkylthio, C₁₋₆ alkylsulfinyl, C₁₋₆ alkylsulfonyl, C₆₋₁₀ arylthio, C₆₋₁₀ arylsulfinyl, C₆₋₁₀ arylsulfonyl, C₆₋₁₀ aryl, C₁₋₆ alkyl(C₆₋₁₀)aryl, and halo(C₆₋₁₀)aryl.

The phrase “one or more substituents”, as used herein, refers to a number of substituents that equals from one to the maximum number of substituents possible based on the number of available bonding sites, provided that the above conditions of stability and chemical feasibility are met. Unless otherwise indicated, an optionally substituted group may have a substituent at each substitutable position of the group, and the substituents may be either the same or different. As used herein, the term “independently selected” means that the same or different values may be selected for multiple instances of a given variable in a single compound.

Unless otherwise explicitly stated, the term “proteasome” is intended to refer to constitutive proteasome, immunoproteasome, or both.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

As used herein, the term “comprises” means “includes, but is not limited to.”

As used herein, the term “patient”, means an animal, preferably a mammal, more preferably a human.

As used herein, the term “cancer” refers to a cellular disorder characterized by uncontrolled or disregulated cell proliferation, decreased cellular differentiation, inappropriate ability to invade surrounding tissue, and/or ability to establish new growth at ectopic sites. The term “cancer” further encompasses primary and metastatic cancers.

As used herein, the term “therapeutically effective amount” means an amount that is sufficient upon appropriate administration to a patient (a) to cause a detectable decrease in the severity of the disorder or disease state being treated; (b) to ameliorate or alleviate the patient's symptoms of the disease or disorder; or (c) to slow or prevent advancement of, or otherwise stabilize or prolong stabilization of, the disorder or disease state being treated (e.g., prevent additional tumor growth of a cancer). It should also be understood that a specific dosage and treatment regimen for any particular patient will depend upon a variety of factors, including the activity of the specific compound employed, the age, body weight, general health, sex, and diet of the patient, time of administration, rate of excretion, drug combinations, the judgment of the treating physician, and the severity of the particular disease being treated.

As used herein, the term “treating nasopharyngeal cancer” means treating a patient having, or at risk of developing or experiencing a recurrence of, nasopharyngeal cancer.

As used herein, the term “IHC” means immunohistochemistry. IHC refers to the process of detecting antigens (e.g., proteins) in cells of a tissue section by exploiting the principle of antibodies binding specifically to antigens in biological tissues.

As used herein the term “NFκB p65 IHC assay” or “Nuclear Factor Kappa-B p65 immunohistochemistry assay” refers to an IHC assay that measures the amount of the NFκB p65 protein in a tissue section of a nasopharyngeal tumor sample by using a antibody or antigen-binding fragment thereof (Fv, Fab, scFv, Fab′ or F(ab′)) that binds to p65 (NFKB3, GenPept Accession No. NP_068810 or an isoform thereof; product of RELA gene, ID 5970; sequences associated with p65 and its isoforms can be found at the website maintained by the National Institute for Biotechnology Information, Bethesda, Md.). p65 antibodies or antibody fragments thereof can be prepared by one of skill in the art or purchased commercially. In some embodiments, the p65 antibody is a rabbit monoclonal antibody. In some embodiments, the p65 antibody is a rabbit polyclonal antibody. In some embodiments, the p65 antibody is a goat polyclonal antibody. The NFκB p65 IHC assay can be an in vitro assay.

Antibodies which bind to NFκB p65 (anti-NFκB p65 antibodies) are readily available from commercial sources; for example, from Cell Signaling Technology, Danvers, Mass.; Invitrogen Corporation, Camarillo Calif.; Santa Cruz Biotechnology, Inc., Santa Cruz, Calif.; Abcam, Cambridge, Mass.; Rockland Immunochemicals, Inc., Gilbertsville, Pa., Novus Biologicals, Littleton, Colo.; or BD Biosciences, San Jose, Calif. Alternatively, an antibody which binds NFκB p65 can be generated by any of a number of methods known to those skilled in the art; such as by immunizing an animal, such as a chicken, mouse, rat, dog, sheep, cow, goat or rabbit with NFκB p65 or a portion thereof, such as a peptide isolated or cleaved and purified from the whole protein, expressed recombinantly as a partial length protein, or synthesized chemically. In some embodiments, the animal is a rabbit. In some embodiments, the animal is a goat. Further details on how to generate and isolate antibodies can be found in reference texts, such as Antibodies: A Laboratory Manual (1988) Harlow and Lane, Cold Spring Harbor Laboratory Press, Cold Spring Harbor, N.Y.

As used herein the term “antibody” is used in the broadest sense and specifically covers full length monoclonal antibodies, immunoglobulins, polyclonal antibodies, multispecific antibodies (e.g. bispecific antibodies) formed from at least two full length antibodies, e.g., each to a different antigen or epitope, and individual antigen binding fragments, including dAbs, scFv, Fab, F(ab)′₂, Fab′, including human, humanized and antibodies from non-human species and recombinant antigen binding forms such as monobodies and diabodies.

Development, i.e., processing for visualization of antibody binding, of a NFκB p65 IHC assay and measurement of NFκB p65 amount can be by a variety of methods known in the art. Development and quantification of antibody binding to NFκB p65 can be through a detectable label. Labeling can be direct or indirect. The label can be colorimetric, fluorescent or radioactive. In some embodiments, an antibody which binds to NFκB p65 directly comprises a detectable label. In other embodiments, development is through a reagent, such as a secondary antibody or biotin-avidin complex, which comprises a label and binds to the NFκB p65-binding antibody.

As used herein the term “H-score” is used to mean an immunohistology score for a nasopharyngeal cancer tumor sample. In an attempt to accurately describe the extent of immunohistochemical staining of a tumor, the degree of IHC staining, if any, in each sub-cellular compartment in tumor cells is captured for each analyte. This algorithm includes capturing the percentage of tumor cells stained at each intensity level. A semi-quantitative intensity scale ranging from 0 for no staining to 3+ for the most intense staining is used. All of this information can be analyzed separately or used to calculate a variable, more continuous than simply Positive versus Negative, called the H-Score. This score is more representative of the staining of the entire tumor on the section. Although given sections may share the same simple intensity score, there is a difference between a 3+ case with only 10% of the cells staining as compared to a 3+ case where greater than 90% of the cells are staining. This difference is easily picked up using H-Score method. An H-Score is typically calculated for staining of each sub-cellular compartment for both normal and tumor cells using the following formula; H-Score=(% cells at 0)*0+(% cells at 1+)*1+(% cells at 2+)*2+(% cells at 3+)*3. Thus, this score produces a continuous variable that ranges from 0 to 300.

For IHC assays, controls may include paraffin section of cell blocks prepared from admixtures of target positive cells and non-target cells (Liu et al. (2002) Am. J. Clin Pathol. 118:216), such as mixtures of cell lines with positive NFκB p65 amounts and peripheral blood leukocytes. These cell blocks can contain mixtures of target cells at the 10%, 30%, 50%, 80%, and 90% levels.

Other controls for assays to measure elevated NFκB p65 amount, such as an NFκB p65 IHC assay, can be a sample of non-cancerous nasopharyngeal tissue. In one embodiment, a sample of non-cancerous nasopharyngeal tissue can be nasopharyngeal tissue obtained in the vicinity of the tumor from the same patient from whom the nasopharyngeal tumor sample is obtained, either in the same tissue specimen or a separate tissue specimen. In another embodiment, a sample of non-cancerous nasopharyngeal tissue can be nasopharyngeal tissue from an animal, such as a human, without nasopharyngeal cancer. Controls for assay performance can include tissues with known high or low amounts of NFκB p65 expression. For example, tissues with B-cell maturation centers, such as tonsils, can have high levels of NFκB p65 expression and can serve as a positive control. Conversely, less active tissues can serve as a negative control. A suitable negative control tissue can be a non-cancerous adult liver sample.

In some embodiments, the H-score is determined manually by a trained pathologist. In some embodiments, the H-score is determined using an automated cellular imaging system and software. In some embodiments, the H-score is determined using a trained pathologist supplemented by an automated scoring method. See for example Choudhury et al., J. Histochem. Cytochem. 58: 95-107 (2010).

A H-score may be given a numerical value between 1 and 300 or may be expressed using values such as low, medium and high. As used herein a low H-score is defined as between 10 and 100; a medium H-score is defined as between 101 and 200; and a high H-score is defined as between 201 and 300.

The level of NFκB p65 in a tumor sample may also be measured by using an anti-NFκB p65 antibody with western blotting and ELISA techniques. For such methods, a lysate is made from a homogenate of tissue or tumor sample. Differential lysis and isolation techniques can separate nuclei from cytoplasm to allow for quantification of nuclear NFκB p65 separately from quantification of cytoplasmic NFκB p65. Homogenates of cells or tissues lysed for such measurements can have appropriate inhibitors, such as protease inhibitors, known in the art to preserve protein structures against degradation. NFκB p65 levels can be quantified on western blots by using software to calculate NFκB p65 band intensity. Some ELISA assays can include the NF-κB DNA response element bound by NFκB p65 to capture NFκB p65 from the lysate for subsequent detection and quantification with the anti-NFκB p65 antibody.

The level of NFkB p65 expression in a tumor sample may also be measured by immunofluorescence, such as of anti-NFκB p65 antibody bound to a tissue section, such as a frozen tissue section or to permeabilized isolated cells.

The level of NFκB p65 expression in a tumor sample may also be measured by RT-PCR using a probe that detects and/or primers that amplify the NFκB p65 mRNA (GenBank NM_021975 or variant thereof, e.g., a nucleic acid encoding any of the sequences selected from the group consisting of SEQ ID NO: 1, SEQ ID NO:2, SEQ ID NO:3, SEQ ID NO:4). RNA can be obtained from either fresh tissue or tumor sample or from paraffin-embedded sections of tumor sample by methods known to those skilled in the art. Expression of NFκB p65 may also be inferred from an analysis of genes targeted with the NFκB response element. This analysis can further include quantification of genes such as NFKB1A and CCND2. See Weichert et al., B. J. Cancer 97:523-530 (2007).

Unless otherwise stated, structures depicted herein are also meant to include compounds which differ only in the presence of one or more isotopically enriched atoms. For example, compounds having the present structure except for the replacement of a hydrogen atom by a deuterium or tritium, or the replacement of a carbon atom by a ¹³C- or ¹⁴C-enriched carbon are within the scope of the invention.

As used herein, the term “boronic acid” refers to a chemical compound containing a —B(OH)₂ moiety. In some embodiments, boronic acid compounds can form oligomeric anhydrides by dehydration of the boronic acid moiety. For example, Snyder et al., J Am. Chem. Soc. 80:3611 (1958), reports oligomeric arylboronic acids.

As used herein, the term “boronic acid anhydride” refers to a chemical compound formed by combination of two or more molecules of a boronic acid compound, with loss of one or more water molecules. When mixed with water, the boronic acid anhydride compound is hydrated to release the free boronic acid compound. In various embodiments, the boronic acid anhydride can comprise two, three, four, or more boronic acid units, and can have a cyclic or linear configuration. Non-limiting examples of oligomeric boronic acid anhydrides of peptide boronic acids compound of the invention are illustrated below:

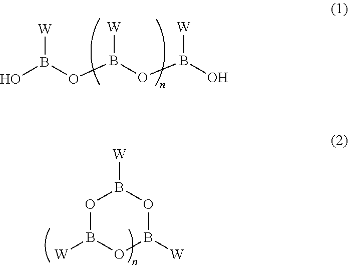

In formulae (1) and (2) directly above, the variable n is an integer from 0 to about 10, preferably 0, 1, 2, 3, or 4. In some embodiments, the boronic acid anhydride compound comprises a cyclic trimer (“boroxine”) of formula (2), wherein n is 1. The variable W has the formula (3):

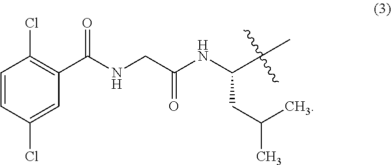 [SMILES: *[C@H](CC(C)C)NC(=O)CNC(=O)c1cc(Cl)ccc1Cl]

In some embodiments, at least 80% of the boronic acid present in the boronic acid anhydride compound exists in a single oligomeric anhydride form. In some embodiments, at least 85%, 90%, 95%, or 99% of the boronic acid present in the boronic acid anhydride compound exists in a single oligomeric anhydride form. In certain preferred embodiments, the boronic acid anhydride compound consists of, or consists essentially of, a boroxine having formula (3).

The boronic acid anhydride compound preferably can be prepared from the corresponding boronic acid by exposure to dehydrating conditions, including, but not limited to, recrystallization, lyophilization, exposure to heat, and/or exposure to a drying agent. Nonlimiting examples of suitable recrystallization solvents include ethyl acetate, dichloromethane, hexanes, ether, acetonitrile, ethanol, and mixtures thereof.

In some embodiments, Z¹ and Z² together form a moiety derived from a boronic acid complexing agent as disclosed in Olhava and Danca, U.S. Pat. Nos. 7,442,830, 7,867,662, and 8,003,819 all of which are herein incorporated by reference in their entirety. For purposes of the invention, the term “boronic acid complexing agent” refers to any compound having at least two functional groups, each of which can form a covalent bond with boron. Nonlimiting examples of suitable functional groups include amino, hydroxyl, and carboxyl. In some embodiments, at least one of the functional groups is a hydroxyl group. The term “moiety derived from a boronic acid complexing agent” refers to a moiety formed by removing the hydrogen atoms from two functional groups of a boronic acid complexing agent.

As used herein, the terms “boronate ester” and “boronic ester” are used interchangeably and refer to a chemical compound containing a —B(Z¹)(Z²) moiety, wherein at least one of Z¹ or Z² is alkoxy, aralkoxy, or aryloxy; or Z¹ and Z² together form a moiety derived from a boronic acid complexing agent having at least one hydroxyl group.

In some embodiments, Z¹ and Z² are each hydroxy and the compound of formula (I) is characterized by formula (II):

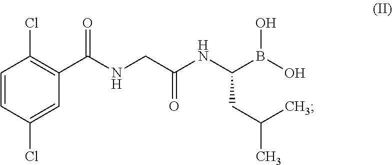 [SMILES: CC(C)C[C@H](NC(=O)CNC(=O)c1cc(Cl)ccc1Cl)B(O)O]

or a pharmaceutically acceptable salt or a pharmaceutical composition or a boronic acid anhydride thereof.

The compound of formula (II), [(1R)-1-({[(2,5-dichlorobenzoyl)amino]acetyl}amino)-3-methylbutyl]boronic acid (MLN2238) is disclosed in Olhava and Danca, U.S. Pat. No. 7,442,830, herein incorporated by reference in its entirety.

In some other embodiments, Z¹ and Z² together form a moiety derived from a compound having at least two hydroxyl groups separated by at least two connecting atoms in a chain or ring, said chain or ring comprising carbon atoms and, optionally, a heteroatom or heteroatoms which can be N, S, or O, wherein the atom attached to boron in each case is an oxygen atom.

As employed herein, the term “compound having at least two hydroxyl groups” refers to any compound having two or more hydroxyl groups. For purposes of the invention, the two hydroxyl groups preferably are separated by at least two connecting atoms, preferably from about 2 to about 5 connecting atoms, more preferably 2 or 3 connecting atoms. For convenience, the term “dihydroxy compound” may be used to refer to a compound having at least two hydroxyl groups, as defined above. Thus, as employed herein, the term “dihydroxy compound” is not intended to be limited to compounds having only two hydroxyl groups. The moiety derived from a compound having at least two hydroxyl groups may be attached to boron by the oxygen atoms of any two of its hydroxyl groups. Preferably, the boron atom, the oxygen atoms attached to boron, and the atoms connecting the two oxygen atoms together form a 5- or 6-membered ring.

For purposes of the present invention, the boronic acid complexing agent preferably is pharmaceutically acceptable, i.e., suitable for administration to humans. In some embodiments, the boronic acid complexing agent is a sugar, as described, e.g., in Plamondon et al., WO 02/059131 and Gupta et al., WO 02/059130. The term “sugar” includes any polyhydroxy carbohydrate moiety, including monosaccharides, disaccharides, polysaccharides, sugar alcohols and amino sugars. In some embodiments, the sugar is a monosaccharide, disaccharide, sugar alcohol, or amino sugar. Non-limiting examples of suitable sugars include glucose, sucrose, fructose, trehalose, mannitol, sorbitol, glucosamine, and N-methylglucosamine. In certain embodiments, the sugar is mannitol or sorbitol. Thus, in the embodiments wherein the sugar is mannitol or sorbitol, Z¹ and Z² together form a moiety of formula C₆H₁₂O₆, wherein the oxygen atoms of the two deprotonated hydroxyl groups form covalent attachments with boron to form a boronate ester compound. In certain embodiments, Z¹ and Z² together form a moiety derived from D-mannitol as disclosed in U.S. Pat. No. 7,442,830, herein incorporated by reference in its entirety.

In some embodiments, the boronic acid complexing agent is an alpha-hydroxycarboxylic acid or a beta-hydroxycarboxylic acid, as described, e.g., in Elliott et al., WO 09/154737, herein incorporated by reference in its entirety. In some embodiments, the boronic acid complexing agent is selected from the group consisting of glycolic acid, malic acid, hexahydromandelic acid, citric acid, 2-hydroxyisobutyric acid, 3-hydroxybutyric acid, mandelic acid, lactic acid, 2-hydroxy-3,3-dimethylbutyric acid, 2-hydroxy-3-methylbutyric acid, 2-hydroxyisocaproic acid, beta-hydroxyisovaleric acid, salicylic acid, tartaric acid, benzilic acid, glucoheptonic acid, maltonic acid, lactobionic acid, galactaric acid, embonic acid, 1-hydroxy-2-naphthoic acid, and 3-hydroxy-2-naphthoic acid. In certain embodiments, the boronic acid complexing agent is citric acid.

In certain embodiments, wherein the alpha-hydroxy carboxylic acid or beta-hydroxy carboxylic acid is citric acid, the compound of formula (I) is characterized by formula (III-A) or (III-B):

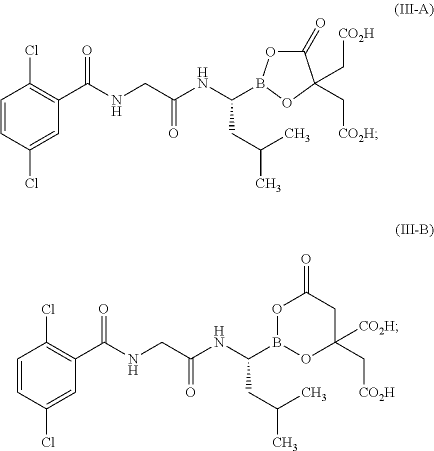 [SMILES: CC(C)C[C@H](NC(=O)CNC(=O)c1cc(Cl)ccc1Cl)B1OC(=O)C(CC(=O)O)(CC(=O)O)O1 ; CC(C)C[C@H](NC(=O)CNC(=O)c1cc(Cl)ccc1Cl)B1OC(=O)CC(CC(=O)O)(C(=O)O)O1]

or a mixture thereof or a pharmaceutical composition thereof.

In certain embodiments, wherein the alpha-hydroxy carboxylic acid or beta-hydroxy carboxylic acid is citric acid, the compound of formula (I) is characterized by formula (III-A):

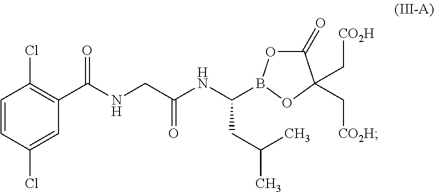 [SMILES: CC(C)C[C@H](NC(=O)CNC(=O)c1cc(Cl)ccc1Cl)B1OC(=O)C(CC(=O)O)(CC(=O)O)O1]

or a pharmaceutical composition thereof.

The compound of formula (III-A), 2,2′-{2-[(1R)-1-({[(2,5-dichlorobenzoyl)amino]acetyl}amino)-3-methylbutyl]-5-oxo-1,3,2-dioxaborolane-4,4-diyl}diacetic acid (MLN9708) is disclosed in Elliott et al., WO 09/154737, herein incorporated by reference in its entirety.

The present invention provides a method of treating nasopharyngeal cancer, comprising administering to a patient in need thereof a therapeutically effective amount of anyone of the compounds of formulas (I), (II), (III-A) or (III-B), or a pharmaceutically acceptable salt or a pharmaceutical composition or a boronic acid anhydride thereof. In some embodiments, the invention provides a method of treating nasopharyngeal cancer, comprising administering to a patient in need thereof a therapeutically effective amount of the compound of formula (III-A) or a pharmaceutical composition thereof.

In some embodiments, the invention provides anyone of the compounds of formulas (I), (II), (III-A) or (III-B), or a pharmaceutically acceptable salt or a pharmaceutical composition or a boronic acid anhydride thereof for use in treating nasopharyngeal cancer. In some embodiments, the invention provides the compound of formulas (III-A), or a pharmaceutical composition thereof for use in treating nasopharyngeal cancer. In some embodiments, the invention provides the use of anyone of the compounds of formulas (I), (II), (III-A) or (III-B), or a pharmaceutically acceptable salt or boronic acid anhydride thereof, for the preparation of a pharmaceutical composition (as described herein) for the treatment of nasopharyngeal cancer. In some embodiments, the invention provides the use of the compound of formula (III-A), or a pharmaceutically acceptable salt, for the preparation of a pharmaceutical composition (as described herein) for the treatment of nasopharyngeal cancer. In some embodiments, the invention provides the use of a therapeutically effective amount of anyone of the compounds of formulas (I), (II), (III-A) or (III-B), or a pharmaceutically acceptable salt or boronic acid anhydride thereof, for the treatment of nasopharyngeal cancer. In some embodiments, the invention provides the use of a therapeutically effective amount of the compound of formula (III-A), or a pharmaceutically acceptable salt thereof, for the treatment of nasopharyngeal cancer.

In some embodiments, the invention provides a method of determining whether to treat a patient with nasopharyngeal cancer with a therapeutically effective amount of anyone of the compounds of formulas (I), (II), (III-A) or (III-B), or a pharmaceutically acceptable salt or a pharmaceutical composition or a boronic acid anhydride thereof, based on identifying the patient with nasopharyngeal cancer as being likely to respond based upon an elevated level of NFκB p65 in the patient's nasopoharyngeal cancer tumor sample. In some embodiments, the invention provides a method of determining whether to treat a patient with nasopharyngeal cancer with a therapeutically effective amount of the compound of formula (III-A), or a pharmaceutical composition thereof based on identifying the patient with nasopharyngeal cancer as being likely to respond based upon an elevated level of NFκB p65 in the patient's nasopoharyngeal cancer tumor sample.

In some embodiments, the invention provides a compound of anyone of formulas (I), (II), (III-A) or (III-B), or a pharmaceutically acceptable salt or a pharmaceutical composition or a boronic acid anhydride thereof, for use in treating nasopharyngeal cancer in a patient with an elevated level of NFκB p65. In some embodiments, the invention provides a compound of anyone of formulas (I), (II), (III-A) or (III-B), or a pharmaceutically acceptable salt or a pharmaceutical composition or a boronic acid anhydride thereof, for use in treating nasopharyngeal cancer in a patient with a H-score of between 201 and 300, as measured by a NFκB p65 IHC assay. In some embodiments, the invention provides a compound of formulas (III-A), or a pharmaceutical composition thereof, for use in treating nasopharyngeal cancer in a patient with a H-score of between 201 and 300, as measured by a NFκB p65 IHC assay.

In some embodiments, the invention provides a compound of anyone of formulas (I), (II), (III-A) or (III-B), or a pharmaceutically acceptable salt or a pharmaceutical composition or a boronic acid anhydride thereof, for use in treating nasopharyngeal cancer in a patient comprising assaying a nasopharyngeal tumor sample from a patient, determining if the patient's nasopharyngeal tumor has an elevated level of NFκB p65, and administering a therapeutically effective amount of the compound of anyone of formulas (I), (II), (III-A) or (III-B), or a pharmaceutically acceptable salt or a pharmaceutical composition or a boronic acid anhydride thereof, if the patient's nasopharyngeal tumor has an elevated level of NFκB p65. In some embodiments, the invention provides a compound of anyone of formulas (I), (II), (III-A) or (III-B), or a pharmaceutically acceptable salt or a pharmaceutical composition or a boronic acid anhydride thereof, for use in treating nasopharyngeal cancer in a patient comprising assaying a nasopharyngeal tumor sample from a patient, determining if the patient's nasopharyngeal tumor has a NFκB p65 level of 201 to 300 as measured by an IHC assay, and administering a therapeutically effective amount of the compound of anyone of formulas (I), (II), (III-A) or (III-B), or a pharmaceutically acceptable salt or a pharmaceutical composition or a boronic acid anhydride thereof, if the patient's nasopharyngeal tumor has a NFκB p65 level of 201 to 300 as measured by an IHC assay. In some embodiments, the invention provides a compound of formula (III-A) or a pharmaceutical composition thereof, for use in treating nasopharyngeal cancer in a patient comprising assaying a nasopharyngeal tumor sample from a patient, determining if the patient's nasopharyngeal tumor has a NFκB p65 level of 201 to 300 as measured by an IHC assay, and administering a therapeutically effective amount of the compound of formula (III-A) or a pharmaceutical composition thereof, if the patient's nasopharyngeal tumor has a NFκB p65 level of 201 to 300 as measured by an IHC assay.

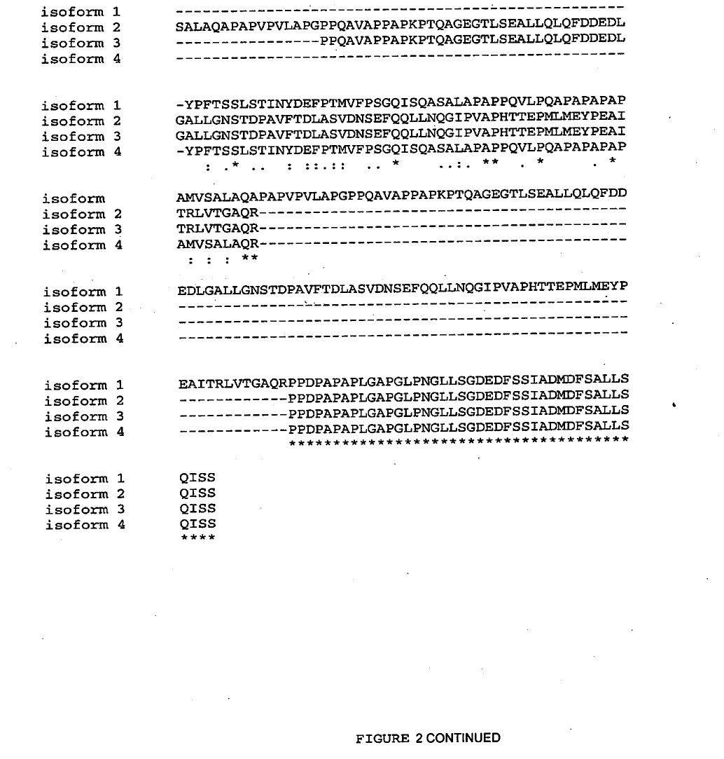

Splice variation of the NFκB p65 gene (ID 5970) results in isoforms of the NFκB p65 protein. Multiple alignment of four NFκB p65 isoforms is shown in FIG. 2. Portions of NFκB p65 which are common to all isoforms are apparent upon review of FIG. 2. In some embodiments, measurement of elevated NFκB p65, such as by the NFκB p65 IHC assay, comprises the use of an antibody which recognizes all isoforms of NFκB p65. For example, the detection antibody can be prepared to bind to a portion of NFκB p65 which is common to all isoforms. For example, the antibody can bind to the carboxy terminal (C-terminal) portion of NFκB p65 or the amino terminal (N-terminal) portion of NFκB p65. As used herein, the C-terminal portion of NFκB p65 consists of about 42 amino acid residues at the NFκB p65 C-terminus. Accordingly, an antibody which detects all isoforms of NFκB p65 can be generated using the sequence of the C-terminal portion, for example, about 10 to about 40 amino acid residues, about 30 to about 40 amino acid residues, at least 15 amino acid residues, at least 20 amino acid residues or at least 25 amino acid residues from the C-terminal portion of NFκB p65. As used herein, the N-terminal portion of NFκB p65 consists of about 150 amino acid residues at the NFκB p65 N-terminus. Accordingly, an antibody which detects all isoforms of NFκB p65 can be generated using the sequence of the N-terminal portion, for example, about 10 to about 150 amino acid residues, about 20 to about 100 amino acid residues, about at least 15 amino acid residues, at least 20 amino acid residues or at least 25 amino acid residues from the N-terminal portion of NFκB p65. Isoform 1 is the longest isoform of NFκB p65 and comprises segments present in all isoforms. Immunization with an isolated NFκB p65 isoform 1 can elicit antibodies which bind all isoforms.

Conversely, comparison of isoform sequences, such as in multiple alignment or pairwise alignment, can indicate regions of NFκB p65 which are not present in other isoforms. Accordingly, an antibody for an assay to measure only a single isoform can be generated to bind to a region, such as a splice junction, which is only in one isoform. Similarly, an antibody can be generated to bind to a region which is in some isoforms, but not others, e.g., a portion of isoform 1, SEQ ID NO: 1 which is not present in SEQ ID NO:2, SEQ ID NO:3 or SEQ ID NO:4, a portion of SEQ ID NOs: 1 and 2 which is not present in SEQ ID NOs:3 or 4, or a portion of SEQ ID NOs: 1, 2 and 3 which is not present in SEQ ID NO:4.

In some embodiments, an assay to measure NFκB p65, such as the NFκB p65 IHC assay, comprises the use of an antibody which binds to a protein with the sequence of SEQ ID NO: 1. In some embodiments, the NFκB p65 assay comprises the use of an antibody which binds to a protein with the sequence of SEQ ID NO:2. In some embodiments, the NFκB p65 assay comprises the use of an antibody which binds to a protein with the sequence of SEQ ID NO:3. In some embodiments, the NFκB p65 assay comprises the use of an antibody which binds to a protein with the sequence of SEQ ID NO:4.

In some embodiments, an assay to measure NFκB p65, such as the NFκB p65 IHC assay measures the amount of SEQ ID NO: 1. In some embodiments, the NFκB p65 assay measures the amount of SEQ ID NO:2. In some embodiments, the NFκB p65 assay measures the amount of SEQ ID NO:3. In some embodiments, the NFκB p65 assay measures the amount of SEQ ID NO:4. In some embodiments, the NFκB p65 assay measures the amount of all isoforms of the 65 kDa subunit of NF-κB. In an embodiment, an NFκB p65 assay measures the total amount of proteins with the sequences of SEQ ID NOs:1, 2, 3 and 4.

Due to variations among human populations, for detecting total NFκB p65, it might be advantageous to use an assay which is not sensitive to variation in NFκB p65 sequence, structure or post-translational modification. Accordingly, in some embodiments, an assay to measure NFκBp65, such as the NFκB p65 IHC assay, comprises the use of polyspecific antibody detection. In some embodiments, the NFκB p65 assay comprises the use of a polyclonal antibody. The polyclonal antibody can be serum or minimally purified, e.g., from serum, milk or yolk, after immunization of an animal with an entire NFκB p65 protein or a portion thereof, the minimal purification to exclude non-immunoglobulin, e.g., serum, milk or yolk, proteins and/or antibodies which bind to proteins other than NFκB p65. In some embodiments, the NFκB p65 assay comprises the use of more than one, such as a pair or a population of antibodies generated against more than one specific portion of NFκB p65, to allow binding to more than one specific portion of NFκB p65.

Another advantage to using an assay, such as NFκB p65 IHC assay, which is not sensitive to variation is due to possible variation in sample preparation methods and sample quality. Since the method contemplates the use of archival samples, some degradation may occur prior to the assay. A balance can be struck between detecting all types or structures of NFκB p65 and keeping a high signal relative to background staining by several art-known immunochemistry, such as IHC, techniques. For example, the practitioner optionally may undertake pre-assay choices and/or IHC assay choices. Examples of pre-assay choices include choice of antibody, the step of eliminating cross-reaction, e.g., by preadsorbtion of the antibody preparation against a protein whose detection is not desired, choice of label or development reagent, and the method of fixation of the biological sample. Examples of IHC assay choices include method or choice of reagent for blocking non-specific binding, and the method or choice of reagent for washing tissue sections. Optimization of conditions for IHC assays is well known in the art, with techniques guides available, for example Buchwalow and Bocker, Immunohistochemistry Basics and Methods (2010, Springer-Verlag, Berlin, Germany).

Upon activation of the cytoplasmic (or cytosolic) NFκB complex comprising p65 and p50, the p65 transfers with p50 to the nucleus. In some embodiments, an assay to measure NFκB p65, such as the NFκB p65 IHC assay, measures the amount of NFκB p65 in the nucleus. In some embodiments, the NFκB p65 assay measures the amount of NFκB p65 in the cytoplasm. In some embodiments, the NFκB p65 assay measures the total (cytoplasmic+nuclear) amount of NFκB p65 in the cell, tumor section or biopsy sample.

In some embodiments, the nasopharyngeal cancer tumor sample is characterized by having elevated levels of NFκB proteins. In some embodiments, the nasopharyngeal cancer tumor sample is characterized by having an elevated level of NFκB p65. In some embodiments, the nasopharyngeal cancer tumor sample is characterized by having a high H-score as measured by a NFκB p65 IHC assay. In some embodiments, the nasopharyngeal cancer tumor sample is characterized by having a H-score as measured by a NFκB p65 IHC assay of between 201 and 300. In some embodiments, the nasopharyngeal cancer tumor sample is characterized by having a H-score as measured by a NFκB p65 IHC assay of between 210 and 300. In some embodiments, the nasopharyngeal cancer tumor sample is characterized by having a H-score as measured by a NFκB p65 IHC assay of between 220 and 300. In some embodiments, the nasopharyngeal cancer tumor sample is characterized by having a H-score as measured by a NFκB p65 IHC assay of between 230 and 300. In some embodiments, the nasopharyngeal cancer tumor sample is characterized by having a H-score as measured by a NFκB p65 IHC assay of between 240 and 300. In some embodiments, the nasopharyngeal cancer tumor sample is characterized by having a H-score as measured by a NFκB p65 IHC assay of between 250 and 300. In some embodiments, the nasopharyngeal cancer tumor sample is characterized by having a H-score as measured by a NFκB p65 IHC assay of between 260 and 300. In some embodiments, the nasopharyngeal cancer tumor sample is characterized by having a H-score as measured by a NFκB p65 IHC assay of between 270 and 300. In some embodiments, the nasopharyngeal cancer tumor sample is characterized by having a H-score as measured by a NFκB p65 IHC assay of between 280 and 300. In some embodiments, the nasopharyngeal cancer tumor sample is characterized by having a H-score as measured by a NFκB p65 IHC assay of between 290 and 300.

In some embodiments, the nasopharyngeal cancer tumor sample is an archival tumor biopsy sample obtained either at or post-diagnosis. In some embodiments, the nasopharyngeal cancer tumor sample is a fresh tumor biopsy. In some embodiments, the nasopharyngeal cancer tumor sample is a fresh tumor biopsy obtained within 14 days of beginning treatment with anyone of the compounds of formula (I), (II), (III-A) or (III-B), or a pharmaceutically acceptable salt or a pharmaceutical composition or a boronic acid anhydride thereof. In some embodiments, the nasopharyngeal cancer tumor sample is a fresh tumor biopsy obtained within 14 days of beginning treatment with the compound of formula (III-A) or a pharmaceutical composition thereof. In some embodiments, the nasopharyngeal cancer tumor sample is a tumor biopsy obtained after 2 to 10 cycles, after about 4 cycles, after about 6 cycles, or after about 9 cycles of treatment with anyone of the compounds of formula (I), (II), (III-A) or (III-B), or a pharmaceutically acceptable salt or a pharmaceutical composition or a boronic acid anhydride thereof. In some embodiments, the nasopharyngeal cancer tumor sample is a tumor biopsy obtained after 2 to 10 cycles, after about 4 cycles, after about 6 cycles, or after about 9 cycles of treatment with the compound of formula (III-A) or a pharmaceutical composition thereof.

Tissue samples to assay for elevated NFκB p65, such as in a NFκB p65 IHC assay, can be obtained from a patient who is a candidate for treatment as described herein or from a commercial source. Commercial sources of tissue samples, such as tumor samples or control samples, include PhenoPath Laboratories, PLLC, Seattle, Wash.; Proteogenix SAS, Obershausbergen, France; and U.S. Biomax, Inc., Rockville, Md.

A sample for use in an assay to measure NFκB p65, such as the NFκB p65 IHC assay, can be a biological sample comprising cells obtained from a patient afflicted with nasopharyngeal cancer. A sample can contain tumor cells or normal cells from the nasopharynx or a mixture of tumor cells and normal cells. A sample comprising nasopharyngeal tumor cells can be obtained from a biopsy of the nasopharyngeal mucosal epithelium or from a nasopharyngeal lymph node. In some embodiments, the nasopharyngeal cancer sample is selected from the group consisting of squamous cell carcinoma, non-keratinizing carcinoma, and undifferentiated carcinoma.

In some embodiments, anyone of the compounds of formulas (I), (II), (III-A) or (III-B), or a pharmaceutically acceptable salt or a pharmaceutical composition or a boronic acid anhydride thereof is administered orally. In some embodiments, the compound of formula (III-A) or a pharmaceutical composition thereof is administered orally. In certain such embodiments, the compound of formula (III-A) or a pharmaceutical composition thereof is administered in one or more capsules. In some embodiments, anyone of the compounds of formulas (I), (II), (III-A) or (III-B), or a pharmaceutically acceptable salt or a pharmaceutical composition or a boronic acid anhydride thereof is administered intravenously. In certain such embodiments, the compound of formula (III-A) or a pharmaceutical composition thereof is administered intravenously.

In some embodiments, anyone of the compounds of formulas (I), (II), (III-A) or (III-B), or a pharmaceutically acceptable salt or a pharmaceutical composition or a boronic acid anhydride thereof is administered on a weekly schedule. In some embodiments, the compound of formula (III-A) or a pharmaceutical composition thereof is administered on a weekly schedule. In some embodiments, the compounds of formulas (I), (II), (III-A) or (III-B), or a pharmaceutically acceptable salt or a pharmaceutical composition or a boronic acid anhydride thereof is administered on days 1, 8, and 15 of a 28-day cycle. In some embodiments, the compound of formula (III-A) or a pharmaceutical composition thereof is administered on days 1, 8, and 15 of a 28-day cycle.

In some embodiments, anyone of the compounds of formulas (I), (II), (III-A) or (III-B), or a pharmaceutically acceptable salt or a pharmaceutical composition or a boronic acid anhydride thereof is administered on a twice-weekly schedule. In some embodiments, the compound of formula (III-A) or a pharmaceutical composition thereof is administered on a twice-weekly schedule. In some embodiments, anyone of the compounds of formulas (I), (II), (III-A) or (III-B), or a pharmaceutically acceptable salt or a pharmaceutical composition or a boronic acid anhydride thereof is administered on days 1, 4, 8, and 11 of a 21-day cycle. In some embodiments, the compound of formula (III-A) or a pharmaceutical composition thereof is administered on days 1, 4, 8, and 11 of a 21-day cycle.

In some embodiments, the amount of the compound of formula (III-A) is about 0.2 mg to about 7.5 mg per dose based on the amount of the compound of formula (II). In some embodiments, the amount of the compound of formula (III-A) is about 1.6 mg to about 5.5 mg per dose based on the amount of the compound of formula (II). In some embodiments, the amount of the compound of formula (III-A) is about 2.3 mg to about 5.5 mg per dose based on the amount of the compound of formula (II).

In some embodiments, anyone of the compounds of formulas (I), (II), (III-A) or (III-B), or a pharmaceutically acceptable salt or a pharmaceutical composition or a boronic acid anhydride thereof is administered in conjunction with another therapeutic modality. In some embodiments, the compound of formula (III-A) or a pharmaceutical composition thereof is administered in conjunction with another therapeutic modality. In some embodiments, the therapeutic modality is one that is normally administered to patients with nasopharyngeal cancer. In some embodiments, the other therapeutic modality is radiotherapy. In some embodiments, the other therapeutic modality is another therapeutic agent. In some embodiments, the other therapeutic modality is radiotherapy and one or more therapeutic agents. The other therapeutic agent may be administered in the same dosage form or as a separate dosage form. When administered as a separate dosage form, the other therapeutic agent may be administered prior to, at the same time as, or following administration of anyone of the compounds of formulas (I), (II), (III-A) or (III-B), or a pharmaceutically acceptable salt or a pharmaceutical composition or a boronic acid anhydride thereof.

Non-limiting examples of therapeutic agents include DNA damaging chemotherapeutic agents which include topoisomerase I inhibitors (e.g., irinotecan, topotecan, camptothecin and analogs or metabolites thereof, and doxorubicin); topoisomerase II inhibitors (e.g., etoposide, teniposide, epirubicin, and daunorubicin); alkylating agents (e.g., melphalan, chlorambucil, busulfan, thiotepa, ifosfamide, carmustine, lomustine, semustine, streptozocin, decarbazine, methotrexate, mitomycin C, and cyclophosphamide); DNA intercalators (e.g., cisplatin, oxaliplatin, and carboplatin); DNA intercalators and free radical generators such as bleomycin; and nucleoside mimetics (e.g., 5-fluorouracil, capecitibine, gemcitabine, fludarabine, cytarabine, mercaptopurine, thioguanine, pentostatin, and hydroxyurea).

Other non-limiting examples of therapeutic agents include chemotherapeutic agents that disrupt cell replication include: paclitaxel, docetaxel, and related analogs; vincristine, vinblastin, and related analogs; thalidomide, lenalidomide, and related analogs (e.g., CC-5013 and CC-4047); protein tyrosine kinase inhibitors (e.g., imatinib mesylate, erlotonib, sorafenib, crizitonib, vemurafenib and gefitinib); proteasome inhibitors (e.g., bortezomib); NF-κB inhibitors, including inhibitors of IκB kinase; antibodies which bind to proteins overexpressed in cancers and thereby downregulate cell replication (e.g., trastuzumab, rituximab, cetuximab, panitumumab, ipilimumab, and bevacizumab); and other inhibitors of proteins or enzymes known to be upregulated, over-expressed or activated in cancers, the inhibition of which downregulates cell replication.

In some embodiments, the therapeutic agent is selected from the group consisting of cisplatin, 5-flurouracil, epirubicin, docetaxel and paclitaxel.

Radiotherapy may be used as another therapeutic modality prior to, at the same time as, or following administration of anyone of the compounds of formulas (I), (II), (III-A) or (III-B), or a pharmaceutically acceptable salt or a pharmaceutical composition or a boronic acid anhydride thereof. In some embodiments, the radiotherapy is external beam radiotherapy. External beam radiotherapy is given as a series of treatments known as fractions. In some such embodiments, the external beam radiotherapy is conformal radiotherapy. In some embodiments, the radiotherapy is internal radiotherapy. Internal radiotherapy uses a radioactive substance sealed in needles, seeds, wires, or catheters that are placed directly into or near the cancer.

### Formulation and Administration

If a pharmaceutically acceptable salt of the compound of formula (I) is utilized in these compositions, the salt preferably is derived from an inorganic or organic acid or base. For reviews of suitable salts, see, e.g., Berge et al, J Pharm. Sci. 66:1-19 (1977) and Remington: The Science and Practice of Pharmacy, 20th Ed., ed. A. Gennaro, Lippincott Williams & Wilkins, 2000.

Nonlimiting examples of suitable acid addition salts include the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate.

Suitable base addition salts include, without limitation, ammonium salts, alkali metal salts, such as lithium, sodium and potassium salts; alkaline earth metal salts, such as calcium and magnesium salts; other multivalent metal salts, such as zinc salts; salts with organic bases, such as dicyclohexylamine, N-methyl-D-glucamine, t-butylamine, ethylene diamine, ethanolamine, and choline; and salts with amino acids such as arginine, lysine, and so forth. In some embodiments, the pharmaceutically acceptable salt is a base addition salt of a boronic acid compound of formula (I), wherein Z¹ and Z² are both hydroxy.

The term “pharmaceutically acceptable carrier” is used herein to refer to a material that is compatible with a recipient subject, preferably a mammal, more preferably a human, and is suitable for delivering an active agent to the target site without terminating the activity of the agent. The toxicity or adverse effects, if any, associated with the carrier preferably are commensurate with a reasonable risk/benefit ratio for the intended use of the active agent.

The terms “carrier”, “adjuvant”, or “vehicle” are used interchangeably herein, and include any and all solvents, diluents, and other liquid vehicles, dispersion or suspension aids, surface active agents, pH modifiers, isotonic agents, thickening or emulsifying agents, preservatives, solid binders, lubricants and the like, as suited to the particular dosage form desired. Remington: The Science and Practice of Pharmacy, 20th Ed., ed. A. Gennaro, Lippincott Williams & Wilkins, 2000 discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, carbonates, magnesium hydroxide and aluminum hydroxide, glycine, sorbic acid, or potassium sorbate, partial glyceride mixtures of saturated vegetable fatty acids, water, pyrogen-free water, salts or electrolytes such as protamine sulfate, disodium hydrogen phosphate, potassium hydrogen phosphate, sodium chloride, and zinc salts, colloidal silica, magnesium trisilicate, polyvinyl pyrrolidone, polyacrylates, waxes, polyethylene-polyoxypropylene-block polymers, wool fat, sugars such as lactose, glucose, sucrose, and mannitol, starches such as corn starch and potato starch, cellulose and its derivatives such as sodium carboxymethyl cellulose, ethyl cellulose and cellulose acetate, powdered tragacanth; malt, gelatin, talc, excipients such as cocoa butter and suppository waxes, oils such as peanut oil, cottonseed oil, safflower oil, sesame oil, olive oil, corn oil and soybean oil, glycols such as propylene glycol and polyethylene glycol, esters such as ethyl oleate and ethyl laurate, agar, alginic acid, isotonic saline, Ringer's solution, alcohols such as ethanol, isopropyl alcohol, hexadecyl alcohol, and glycerol, cyclodextrins such as hydroxypropyl β-cyclodextrin and sulfobutylether β-cyclodextrin, lubricants such as sodium lauryl sulfate and magnesium stearate, petroleum hydrocarbons such as mineral oil and petrolatum. Coloring agents, releasing agents, coating agents, sweetening, flavoring and perfuming agents, preservatives and antioxidants can also be present in the composition, according to the judgment of the formulator.

The pharmaceutical compositions utilized in the invention can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions.

The pharmaceutical compositions utilized in the invention are formulated for pharmaceutical administration to a mammal, preferably a human being. Such pharmaceutical compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intravenously, or subcutaneously. The formulations of the invention may be designed to be short-acting, fast-releasing, or long-acting. Still further, compounds can be administered in a local rather than systemic means, such as administration (e.g., by injection) at a tumor site.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, cyclodextrins, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Compositions formulated for parenteral administration may be injected by bolus injection or by timed push, or may be administered by continuous infusion.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents such as phosphates or carbonates.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

In some embodiments, the compound of formula (I) is administered orally. In some embodiments, the compound of formula (III-A) or a pharmaceutical composition thereof is administered orally. In some such embodiments, a pharmaceutical composition of the compound of formula (III-A) is prepared in gelatin capsules as described in Elliott et al., WO 09/154737, herein incorporated by reference in its entirety. In some embodiments, the pharmaceutical composition comprises the compound of formula (III-A) or a crystalline form thereof, a filler, optionally a lubricant, optionally a flow-aid and optionally a buffer. In some embodiments, the pharmaceutical composition comprises the compound of formula (III-A) or a crystalline form thereof, a filler, a lubricant, and a flow-aid. In some embodiments, the pharmaceutical composition comprises about 0.2% to about 12% of the compound of formula (III-A), or a crystalline form thereof, about 76.5% to about 99.8% of a filler, optionally up to about 1.5% of a lubricant, and optionally up to about 5% of a flow-aid. The oral pharmaceutical compositions can be prepared by methods described in Elliott et al., WO 09/154737, herein incorporated by reference in its entirety.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In some embodiments, the compound of formula (I) is administered intravenously. In some such embodiments, the compound of formula (I) wherein Z¹ and Z² together form a moiety derived from a boronic acid complexing agent can be prepared in the form of a lyophilized powder, as described in Plamondon et al., WO 02/059131, herein incorporated by reference in its entirety or Elliott et al., WO 09/154737, herein incorporated by reference in its entirety. In some embodiments, the lyophilized powder also comprises free boronic acid complexing agent. Preferably, the free boronic acid complexing agent and the compound of formula (I) are present in the mixture in a molar ratio ranging from about 0.5:1 to about 100:1, more preferably from about 5:1 to about 100:1. In various embodiments, the lyophilized powder comprises free boronic acid complexing agent and the corresponding boronate ester in a molar ratio ranging from about 10:1 to about 100:1, from about 20:1 to about 100:1, or from about 40:1 to about 100:1.

In some embodiments, the lyophilized powder comprises boronic acid complexing agent and a compound of formula (I), substantially free of other components. However, the composition can further comprise one or more other pharmaceutically acceptable excipients, carriers, diluents, fillers, salts, buffers, bulking agents, stabilizers, solubilizers, and other materials well known in the art. The preparation of pharmaceutically acceptable formulations containing these materials is described in, e.g., Remington: The Science and Practice of Pharmacy, 20th Ed., ed. A. Gennaro, Lippincott Williams & Wilkins, 2000, or latest edition. In some embodiments, the pharmaceutical composition comprises a compound of formula (I), a bulking agent, and a buffer. In some embodiments, the pharmaceutical composition comprises a compound of formula (III-A), a bulking agent, and a buffer.

The lyophilized powder comprising the compound of formula (I) or formula (III-A) can be prepared according to the methods described in Plamondon et al., WO 02/059131, herein incorporated by reference in its entirety or Elliott et al., WO 09/154737, herein incorporated by reference in its entirety In some embodiments, the method for preparing the lyophilized powder comprises: (a) preparing an aqueous mixture comprising a boronic acid compound of formula (I), wherein Z¹ and Z² are each hydroxy, and a boronic acid complexing agent; and (b) lyophilizing the mixture. In some embodiments, the method for preparing the lyophilized powder comprises: (a) preparing an aqueous mixture comprising the compound of formula (III-A), a bulking agent, and a buffer; and (b) lyophilizing the mixture.

The lyophilized powder preferably is reconstituted by adding an aqueous solvent suitable for pharmaceutical administrations. Examples of suitable reconstitution solvents include, without limitation, water, saline, and phosphate buffered saline (PBS). Preferably, the lyophilized powder is reconstituted with normal (0.9%) saline. Upon reconstitution, an equilibrium is established between a boronate ester compound and the corresponding free boronic acid compound. In some embodiments, equilibrium is reached quickly, e.g., within 10-15 minutes, after the addition of aqueous medium. The relative concentrations of boronate ester and boronic acid present at equilibrium is dependent upon parameters such as, e.g., the pH of the solution, temperature, the nature of the boronic acid complexing agent, and the ratio of boronic acid complexing agent to boronate ester compound present in the lyophilized powder.

The pharmaceutical compositions utilized in the present invention preferably are formulated for administration to a patient having, or at risk of developing or experiencing a recurrence of, nasopharyngeal cancer. Preferred pharmaceutical compositions utilized in the present invention are those formulated for oral, intravenous, or subcutaneous administration. Any of the above dosage forms containing a therapeutically effective amount of a compound of formula (I) are well within the bounds of routine experimentation and within the scope of the present invention. In some embodiments, the pharmaceutical composition utilized in the present invention may further comprise another therapeutic agent.

The amount of additional therapeutic agent present in a composition of this invention typically will be no more than the amount that would normally be administered in a composition comprising that therapeutic agent as the only active agent. Preferably, the amount of additional therapeutic agent will range from about 50% to about 100% of the amount normally present in a composition comprising that agent as the only therapeutically active agent.

In order that this invention be more fully understood, the following preparative and testing examples are set forth. These examples illustrate how to make or test specific compounds, and are not to be construed as limiting the scope of the invention in any way.

### Examples

### Example 1: Preparation of Compounds and Pharmaceutical Compositions

The compound of formula (II), [(1R)-1-({[(2,5-dichlorobenzoyl)amino]acetyl}amino)-3-methylbutyl]boronic acid, is prepared by methods disclosed in Olhava and Danca, U.S. Pat. No. 7,442,830, herein incorporated by reference in its entirety. The compound of formula (III-A), 2,2′-{2-[(1R)-1-({[(2,5-dichlorobenzoyl)amino]acetyl}amino)-3-methylbutyl]-5-oxo-1,3,2-dioxaborolane-4,4-diyl}diacetic acid, is prepared by methods disclosed in Elliott et al., WO 09/154737, herein incorporated by reference in its entirety. An oral capsule formulation of the compound of formula (III-A) is prepared by methods disclosed in Elliott et al., WO 09/154737, herein incorporated by reference in its entirety. An IV formulation of the compound of formula (III-A) is prepared by methods disclosed in Elliott et al., WO 09/154737, herein incorporated by reference in its entirety. A lyophilized formulation of the compound of formula (III-A) suitable for reconstitution into an IV formulation is prepared by methods disclosed in Elliott et al., WO 09/154737, herein incorporated by reference in its entirety.

### Example 2: Measurement of Cytosolic NFκB p65 IHC Assay H-Score—Assay 1

A previously prepared formalin fixed paraffin embedded (FFPE) immunohistochemistry (IHC) slide is deparaffinized using xylene and graded alcohols on a Sakura DRS slide stainer, and then incubated for 5 minutes in 3% H₂O₂. The slide is treated with steam for 30 minutes in a citrate buffer at pH6. The slide is then treated with a 1:50 solution of a NFκB p65 antigen rabbit monoclonal antibody [Cell Signaling Technologies, clone (C₂₂B4) #4764] overnight in a humid chamber. After overnight incubation the slide is washed and placed on a Dako Autostainer and developed with a secondary antibody (Ultravision, Thermo Fisher) and DAB+ substrate (Dako). After development, the slide is washed in de-ionized water, counterstained using Meyer's hematoxylin, dehydrated and coverslipped.

Each slide is evaluated by the pathologist for nuclear and cytoplasmic signal including intensity of staining (0-3+) and percentage of positive cells in deciles to determine an H-score.

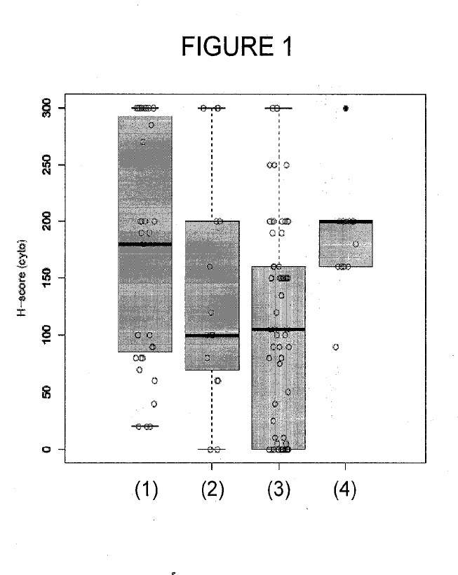

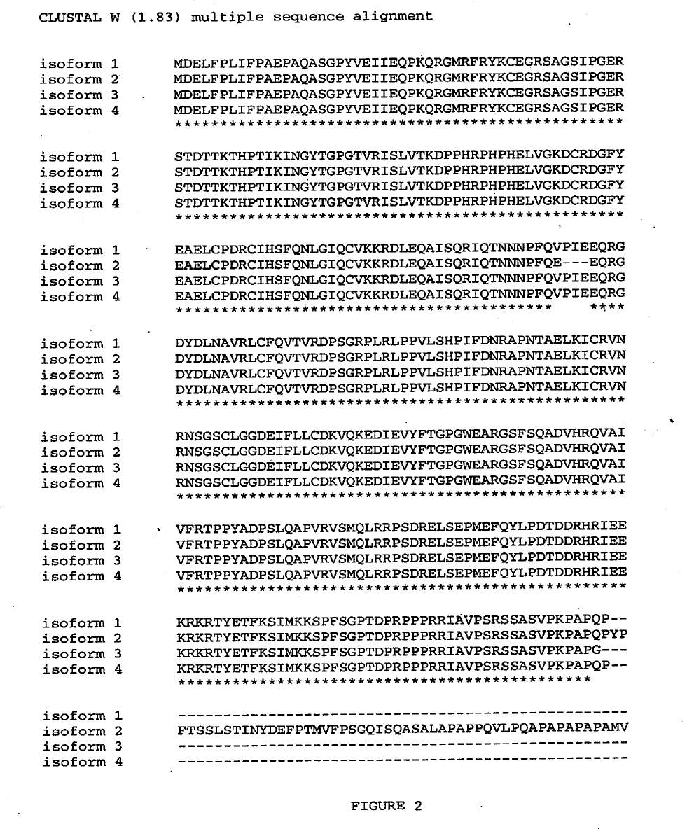

FIG. 1 displays the results of the above assay on the following:

(1) Tissue Microarray (obtained from US Biomax, Inc.) of primary nasopharyngeal cancer (n=35)
(2) Tissue Microarray of metastatic nasopharyngeal cancer (obtained from US Biomax, Inc.) (n=15)
(3) Tissue Microarray of other histologies of head and neck cancers (obtained from US Biomax, Inc.) (n=68)
(4) Archival primary tumor biopsy samples of patients with head and neck squamous cell cancers (n=14). All samples were obtained under the appropriate informed consent. The patients were administered the compound of formula (III-A) on a twice-weekly schedule (days 1, 4, 8 and 11 of a 21-day cycle) as an IV injection.

Of the primary tumor samples in (4), there were three patients with nasopharyngeal cancer. Patient 1 with metastatic nasopharyngeal cancer of a squamous cell type had a durable partial response; Patient 2's disease progressed within 4 cycles; and Patient 3 had progressive disease. These three patients showed the following NFκB p65 IHC assay H-scores (Table 1).

TABLE 1
NFκB p65 IHC assay H-score values for patients with nasopharyngeal
cancer

| | Responder/ | |
|---|---|---|
| Patient | Non-responder | NFκB p65 IHC assay H-score |
| Patient 1 | Responder | 300 |
| Patient 2 | Non-responder | 200 |
| Patient 3 | Non-responder | 160 |

### Example 3: Measurement of Cytosolic NFκB p65 IHC Assay H-Score—Assay 2

A previously prepared FFPE IHC slide is deparaffinized using xylene and graded alcohols on a Sakura DRS slide stainer, and then incubated for 5 minutes in 3% H₂O₂. The slide is treated with steam for 30 minutes in a citrate buffer at pH6. The slide is then treated with a 1:200 solution of a NFκB p65 antigen goat polyclonal antibody [Santa Cruz, sc-372-G] for 1 hour on a Dako Autostainer. After 1 hour incubation the slide is developed with a secondary antibody (Bio-goat Elite (Vector) and DAB+ substrate (Dako). After development, the slide is washed in de-ionized water, counterstained using Meyer's hematoxylin, dehydrated and coverslipped.

Each slide is evaluated by the pathologist for nuclear and cytoplasmic signal including intensity of staining (0-3+) and percentage of positive cells in deciles to determine an H-score.

### Example 4: Measurement of Cytosolic NFκB p65 IHC Assay H-Score—Assay 3

A previously prepared FFPE IHC slide is deparaffinized using xylene and graded alcohols on a Sakura DRS slide stainer, and then incubated for 5 minutes in 3% H₂O₂. The slide is treated in a microwave pressure cooker for 8 minutes in a citrate buffer at pH6. The slide is then treated with a 1:200 solution of a NFκB p65 antigen rabbit polyclonal antibody [Invitrogen, 51-0500] for 1 hour on a Dako Autostainer. After 1 hour incubation the slide is developed with a secondary antibody (Ultravision, Thermo Fisher) and DAB+ substrate (Dako). After development, the slide is washed in de-ionized water, counterstained using Meyer's hematoxylin, dehydrated and coverslipped.

Each slide is evaluated by the pathologist for nuclear and cytoplasmic signal including intensity of staining (0-3+) and percentage of positive cells in deciles to determine an H-score.

### Example 5: Comparison of Section Size for Measurement of NFκB p65

Tissue microarray sections and whole nasopharyngeal tumor sections from commercial sources were assayed by the method described in Example 2. The prevalence of samples with an H score >200 were 35% for tissue microarray samples (n=20) and 31% for whole tissue sections (n=35).

### Example 6: Measurement of NFκB p65 on Whole Tissue Sections

Whole nasopharyngeal tumor sections from commercial sources were assayed by the method described in Example 2.

On a set which expanded the set of whole section samples used in Example 5 to 70 samples overall, the nasopharyngeal NFκB p65 expression frequency was 48.6% (95% CI: 37.1%-60%) (n=70).

While the foregoing invention has been described in some detail for purposes of clarity and understanding, these particular embodiments are to be considered as illustrative and not restrictive. It will be appreciated by one skilled in the art from a reading of this disclosure that various changes in form and detail can be made without departing from the true scope of the invention, which is to be defined by the appended claims rather than by the specific embodiments.

The patent and scientific literature referred to herein establishes knowledge that is available to those with skill in the art. Unless otherwise defined, all technical and scientific terms used herein have the same meaning as commonly understood by one of ordinary skill in the art to which this invention belongs. The issued patents, applications, and references that are cited herein are hereby incorporated by reference to the same extent as if each was specifically and individually indicated to be incorporated by reference. In the case of inconsistencies, the present disclosure, including definitions, will control.

1. A method of treating nasopharyngeal cancer, comprising administering a therapeutically effective amount of the compound of formula (III-A): 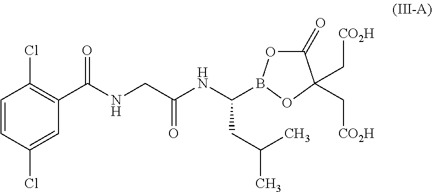 [SMILES: CC(C)C[C@H](NC(=O)CNC(=O)c1cc(Cl)ccc1Cl)B1OC(=O)C(CC(=O)O)(CC(=O)O)O1]
or a pharmaceutically acceptable salt or a pharmaceutical composition thereof;
to a patient whose nasopharyngeal cancer tumor sample is characterized by having a H-score of 300, as measured by a nuclear factor kappa-B (NFκB) p65 immunohistochemistry (IHC) assay.

2. (canceled)

3. (canceled)

4. The method of claim 1, wherein the NFκB p65 IHC assay comprises the use of an antibody which binds to a protein with the sequence of SEQ ID NO: 1.

5. The method of claim 1, wherein the NFκB p65 IHC assay measures the total amount of proteins with the sequences of SEQ ID NOs: 1, 2, 3 and 4.

6. The method of claim 1, wherein the NFκB p65 IHC assay measures the amount of the NFκB p65 present in the cytoplasm.

7. The method of claim 1, wherein the compound of formula (III-A) is administered intravenously.

8. The method of claim 1, wherein the compound of formula (III-A) is administered orally.

9. The method of claim 1, wherein the compound of formula (III-A) is administered on days 1, 8, and 15 of a 28-day cycle.

10. The method of claim 1, wherein the compound of formula (III-A) is administered on days 1, 4, 8, and 11 of a 21-day cycle.

11. (canceled)

12. (canceled)

13. (canceled)

14. (canceled)

15. (canceled)

16. (canceled)

17. (canceled)

18. (canceled)

19. The method of claim 8, wherein the compound of formula (III-A) is administered in one or more capsules.

20. (canceled)

21. (canceled)

22. The method of claim 1, wherein the amount of the compound of formula (III-A) is about 2.3 mg to about 5.5 mg based on the amount of the compound of formula (II).

23. A method for determining whether to treat a patient with nasopharyngeal cancer with a compound of formula (III-A): 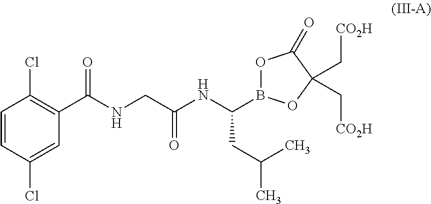 [SMILES: CC(C)C[C@H](NC(=O)CNC(=O)c1cc(Cl)ccc1Cl)B1OC(=O)C(CC(=O)O)(CC(=O)O)O1]
or a pharmaceutically acceptable salt or a pharmaceutical composition thereof;
comprising:
a) measuring the level of NFkB p65 in a nasopharyngeal cancer tumor sample from the patient as a H-score, wherein the H-score is determined by a NFκB p65 IHC assay; and
b) determining to treat the patient with a therapeutically effective amount of the compound of formula (II-A) or a pharmaceutically acceptable salt or a pharmaceutical composition thereof, if the nasopharyngeal cancer tumor sample is characterized by having a H-score of 300 as measured by a NFκB p65 IHC assay.

24. (canceled)

25. (canceled)

26. The method of claim 23, wherein the NFκB p65 IHC assay comprises the use of an antibody which binds to a protein with the sequence of SEQ ID NO: 1.

27. The method of claim 23, wherein the NFκB p65 IHC assay measures the total amount of proteins with the sequences of SEQ ID NOs:1, 2, 3 and 4.

28. The method of claim 23, wherein the NFκB p65 IHC assay measures the amount of the NFκB p65 present in the cytoplasm.

29. The method of claim 23, wherein the compound of formula (III-A) is administered intravenously.

30. The method of claim 23, wherein the compound of formula (III-A) is administered orally.

31. The method of claim 23, wherein the compound of formula (III-A) is administered on days 1, 8, and 15 of a 28-day cycle.

32. The method of claim 23, wherein the compound of formula (III-A) is administered on days 1, 4, 8, and 11 of a 21-day cycle.

33. (canceled)

34. (canceled)

35. (canceled)

36. (canceled)

37. (canceled)

38. (canceled)

39. (canceled)

40. (canceled)

41. The method of claim 23, wherein the compound of formula (III-A) is administered in one or more capsules.

42. (canceled)

43. (canceled)

44. The method of claim 23, wherein the amount of the compound of formula (III-A) is about 2.3 mg to about 5.5 mg based on the amount of the compound of formula (II).
